(19) United States
(12) Patent Application Publication
(10) Pub. No.: US 2015/0127072 A1
(43) Pub. Date: May 7, 2015

(54) DEVICE FOR EMITTING LIGHT BY MEANS OF LEDS OF THE CONTINUOUS AND PULSED TYPE FOR AESTHETIC AND THERAPEUTIC TREATMENTS

(71) Applicant: ESPANSIONE MARKETING S.P.A., Argelato (IT)

(72) Inventor: Rodolfo POMAR, Pianoro (IT)

(73) Assignee: ESPANSIONE MARKETING S.P.A., ARGELATO (IT)

(21) Appl. No.: 14/074,149

(22) Filed: Nov. 7, 2013

(51) Int. Cl. A61N 5/06

(52) U.S. Cl. CPC A61N 5/0616; A61N 2005/0628
USPC 607/90

(57) ABSTRACT

A device for emitting light by means of LEDs of the continuous and pulsed type for aesthetic and therapeutic treatments, of the type comprising an electric power supply circuit which is connected to a respective external control and management unit and at least one LED controlled by the circuit and oriented toward a surface that is intended to face the skin of the patient in conditions for use, the device having a platelike shape; the at least one LED being arranged on a laminar accommodation element that is arranged at a constant distance from the surface intended to face the skin of the patient in conditions for use; the electric circuit comprising at least one apparatus for adjusting the electric power supply values of the at least one LED controlled by the control and management unit in order to set the power of the light beam emitted by the at least one LED.

The present invention relates to a device for emitting light by means of LEDs of the continuous and pulsed type for aesthetic and therapeutic treatments of the skin and scalp.

### BACKGROUND OF THE INVENTION

It is known to resort to handpieces and apparatuses for the treatment of the skin and scalp which comprise irradiation sources (such as xenon lamps, halogen lamps, LEDs, discharge lamps, incandescent lamps and the like).

In many of these constructive solutions it is impossible to arrange in proximity the light source and the skin of the patient, since the light source generates large amounts of heat that might cause burns in said patient.

Constructive solutions that adopt cold light sources (or keep their temperature, even during operation, below threshold values, therefore at temperatures that are not dangerous for the patient) are however becoming increasingly established in relation to the reduction of the risk for the patient and therefore also to greater simplicity of use for the operator.

Furthermore, the aesthetic treatments that use LEDs as light sources are proving themselves particularly efficient for overcoming various skin problems (acne, impure skin, loss of tone and/or toning, wrinkles, dermatitis and others).

These treatments ensure the obtainment of optimum results when the distance of the light sources from the skin and the power of the beam emitted by the source are correlated according to formulas that are specific for each type of treatment apparatus.

Since the surface of the skin of the patient is not perfectly flat (it has instead different curvatures and physiological inflections, which are particularly evident in the face, in the hands, etc.), the power of the emitted beam is selected in relation to an average distance between the sources and the skin of the patient.

Therefore, some portions of skin are subjected to the ideal treatment and others are overexposed (i.e., struck by a beam with higher than ideal power) because they are too close to the source and others will be underexposed (i.e., struck by a beam with lower than ideal power) because they are too far from the source.

For this reason, treatments that can be applied with apparatuses of the known type are generally scarcely efficient, offering poor results which in some cases do not allow to overcome the problem that is the subject of the treatment.

### SUMMARY OF THE INVENTION

The aim of the present invention is to solve the problems described above, proposing a device for emitting light by means of LEDs of the continuous and pulsed type for aesthetic and therapeutic treatments that allows to perform a uniform treatment of the entire skin surface that it faces.

Within this aim, an object of the invention is to propose a device for emitting light by means of LEDs of the continuous and pulsed type for aesthetic and therapeutic treatments that is particularly efficient and safe for the patient.

Another object of the invention is to propose a device for emitting light by means of LEDs of the continuous and pulsed type for aesthetic and therapeutic treatments that is simple to use for the operator.

A further object of the present invention is to provide a device for emitting light by means of LEDs of the continuous and pulsed type for aesthetic and therapeutic treatments that has modest costs, is relatively simple to provide in practice and is safe in application.

This aim and these objects are achieved by a device for emitting light by means of LEDs of the continuous and pulsed type for aesthetic and therapeutic treatments, of the type comprising an electric power supply circuit which is connected to a respective external control and management unit, at least one LED controlled by said circuit and oriented toward a surface that is intended to face the skin of the patient in conditions for use, characterized in that it has a platelike shape, said at least one LED being arranged on a laminar accommodation element that is arranged at a constant distance from the surface intended to face the skin of the patient in conditions for use, said electric circuit comprising at least one apparatus for adjusting the electric power supply values of said at least one LED controlled by said control and management unit in order to set the power of the light beam emitted by said at least one LED.

This aim and these objects are also achieved by means of an apparatus for aesthetic and therapeutic treatments for the type comprising a control and management unit, at least one cable for connection to a treatment device provided with an electric power supply circuit for at least one LED that is oriented toward the surface of said device that is intended to face the skin of the patient in conditions for use, characterized in that said device is platelike and said at least one LED is arranged on a laminar accommodation element that is arranged at a constant distance from the surface that is intended to face the skin of the patient in conditions for use, said electric circuit comprising at least one apparatus for adjusting the electric power supply values of said at least one LED controlled by said control and management unit, in order to set the power of the light beam emitted by said at least one LED as a function of operating parameters contained within memory means of said unit.

### BRIEF DESCRIPTION OF THE DRAWINGS

Further characteristics and advantages of the invention will become better apparent from the description of a preferred but not exclusive embodiment of the device for emitting light by means of LEDs of the continuous and pulsed type for aesthetic and therapeutic treatments, illustrated by way of nonlimiting example in the accompanying drawings, wherein:

FIG. 1 is a schematic perspective view of an embodiment of a device for emitting light by means of LEDs of the continuous and pulsed type for aesthetic and therapeutic treatments according to the invention;

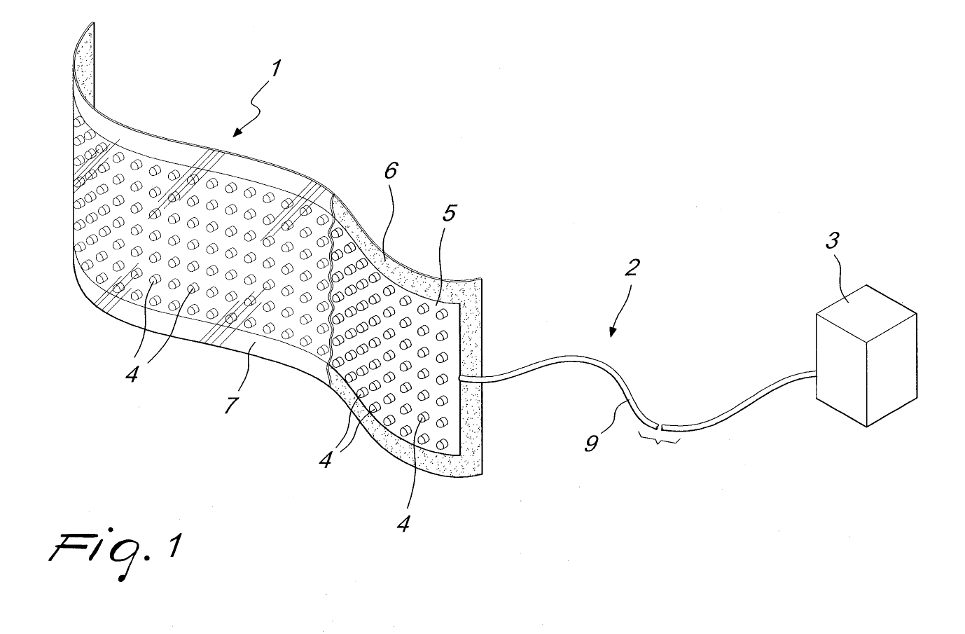

FIG. 2 is a sectional view, taken along a transverse plane, of a device for emitting light by means of LEDs of the continuous and pulsed type for aesthetic and therapeutic treatments according to the invention;

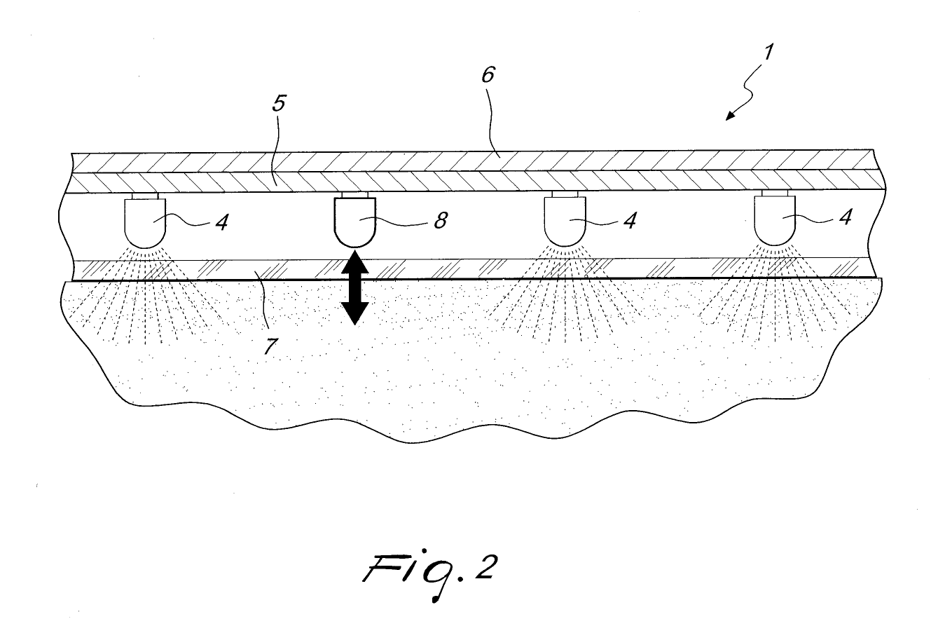

FIG. 3 is a schematic perspective view of a further possible constructive solution of a device for emitting light by means of LEDs of the continuous and pulsed type for aesthetic and therapeutic treatments according to the invention;

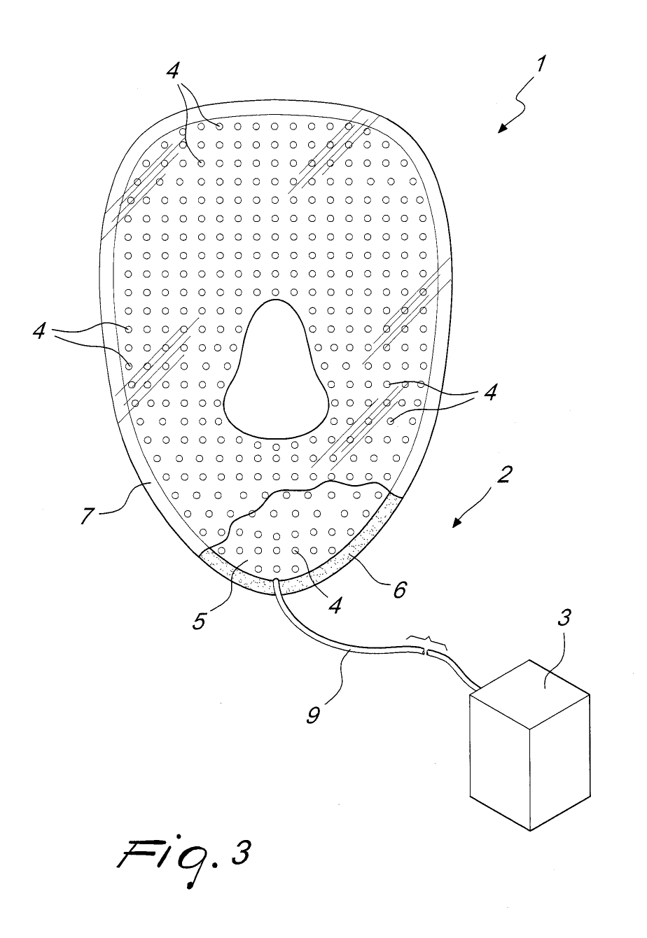

FIG. 4 is a schematic perspective view of a further possible constructive solution of a device for emitting light by means of LEDs of the continuous and pulsed type for aesthetic and therapeutic treatments according to the invention.

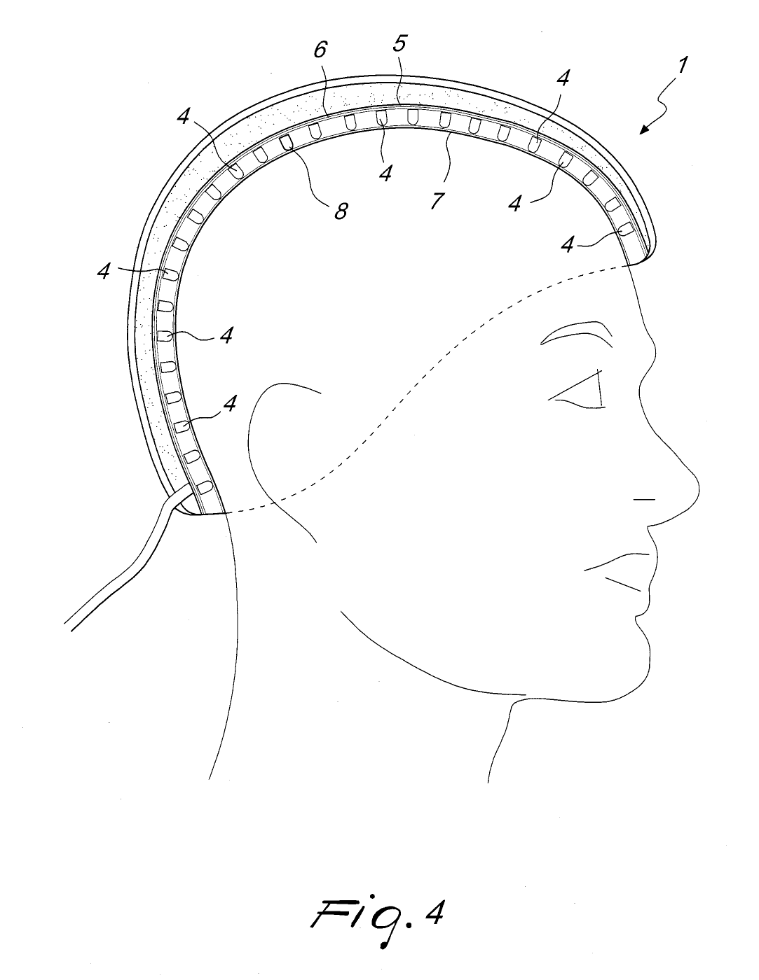

### DESCRIPTION OF THE PREFERRED EMBODIMENTS

With particular reference to the above cited figures, the reference numeral 1 generally designates a device for emitting light by means of LEDs of the continuous and pulsed type for aesthetic and therapeutic treatments and the reference numeral 2 generally designates an apparatus for aesthetic treatments that comprises the device 1.

The device 1 comprises an electric power supply circuit connected to a respective external control and management unit 3 and at least one LED controlled by the circuit and oriented toward a surface that is intended to face the skin of the patient in conditions for use.

The device 1 has a platelike shape and can be made of rigid or flexible material depending on the requirements of application.

The at least one LED 4 is arranged on a laminar accommodation element 5, which is arranged at a constant distance from the surface that is intended to face the skin of the patient in conditions for use.

The electric circuit comprises at least one apparatus for adjusting the electric power supply values of the at least one LED 4 controlled by the control and management unit 3 in order to set the power of the light beam emitted by the at least one LED 4.

In this manner, in other words, it is possible to adjust the power of the light beam emitted by the LED 4 (or by the plurality of LEDs 4) as a function of the specific requirements of application (i.e., as a function of the characteristics of the skin of the patient and of the skin dysfunction/pathology/irritation on which one intends to act).

According to a particular constructive solution of unquestionable practical interest, the electric circuit can comprise preferably a lamina for accommodating the respective components, comprising the at least one LED 4 and the at least one adjustment apparatus (in this case coinciding with the laminar element cited earlier).

The accommodation lamina is provided with surface conducting paths for the functional connection of the various components.

In particular, the lamina can be an ordinary printed circuit, a board for electronic circuits (normally known as PCB, printed circuit board), a flat support made of fiberglass and the like.

With reference to a particularly efficient application solution, the circuit can be interposed between an external panel 6 and a plate 7 that lies on the surface that is intended to face the skin of the patient in conditions for use.

The plate 7 is preferably made of material that is at least partially transparent to the radiation emitted by the at least one LED 4.

The external panel 6 instead is preferably made of opaque material so that the electric circuit and its components are not visible from the outside.

It should be pointed out conveniently that at least one component chosen among the lamina 5, the panel 6 and the plate 7 is made of flexible material.

This allows to arrange the device 1 on the skin of the patient so as to follow its physiological curves, thus ensuring that the correct distance of each LED 4 from the skin of said patient is maintained.

According to a particular automated constructive solution, the device 1 may comprise furthermore a sensor 8 that is intended to detect the light radiation reflected by the skin of the patient.

The control and management unit 3, as a function of the signal emitted by the sensor 8 following the detection of reflected light as a consequence of a test emission of light radiation by the at least one LED 4, sets the power of the light beam emitted by the at least one LED 4 (in the subsequent treatments) by means of the apparatus for adjusting the electric power supply values of the at least one LED 4.

In practice, it is therefore possible to emit a light pulse when the device 1 already faces the skin of the patient.

The sensor 8 (which can be of the type of a photodiode, a photocell, a photovoltaic cell and the like) detects a reflected light value, emitting a signal that is proportional thereto.

This signal indicates to the control and management unit 3 the skin type of the patient: very fair skins, dark skins, tanned skins, presence of irritations, etc.

As a function of the skin type of the patient (detected in this case by means of the sensor 8) and of the type of treatment that it is necessary to perform (in order to contrast acne, reduce wrinkles, cause reabsorption of an irritation), the unit 3 can then establish which is the suitable power of the beam that will be emitted by the LEDs 4.

If one adopts a device 1 without the sensor 8, the operator shall have to estimate the skin type of the patient in order to select the respective phototype and the corresponding characteristics on a control panel of the unit 3.

It is believed important to specify that the LEDs 4 are preferably a plurality, distributed on the laminar accommodation element 5 arranged at a constant distance from the surface intended to face the skin of the patient in conditions for use.

The distribution of the LEDs 4 can be uniform and equidistant or localized in predefined regions, in order to meet the specific requirements of application of each possible treatment.

According to a particular and exemplary constructive solution of unquestionable interest in practice and in application, the device 1 preferably can have a shape and dimensions that are complementary to those of a specific part of the human body for which it is intended.

For example, it is possible to provide a mask that reproduces the shape of the face, a three-dimensional contour that reproduces the shape of the nose, of the region around the eye (comprising the cheekbone and eyebrow), of a hand, of the cranium (for scalp treatments), of a wrist and/or of any other part of the body.

It is specified furthermore that the protection of the present invention also extends to an apparatus 2 for aesthetic treatments that comprises a control and management unit 3 and at least one device 1.

The control and management unit 3 comprises at least one cable 9 for connection to a treatment device 1 provided with an electric power supply circuit for at least one LED 4 that is oriented toward a surface of said device 1 that is intended to face the skin of the patient in conditions for use.

The device 1 has a preferably platelike shape.

The at least one LED 4 is arranged on a laminar accommodation element 5 arranged at a constant distance from the surface that is intended to face the skin of the patient in conditions for use.

The electric circuit comprises at least one apparatus for adjusting the electric power supply values of the at least one LED 4 controlled by the control and management unit 3, in order to set the power of the light beam emitted by the at least one LED 4 as a function of operating parameters contained within memory means of the unit 3.

The unit 3 comprises the memory means intended to collect the treatment protocols related to all the skin problems for which the apparatus 2 provided with the device 1 is intended.

Each protocol can be modified (in terms of power of the emitted beam and duration of the emission) as a function of the characteristics of the skin of the patient (phototype, inflammations in progress, specific pathologies).

It is specified that the control and management unit 3 can comprise for this purpose a selector of a plurality of different treatment protocols related to specific skin problems and to specific skin types of the patient, said protocols being stored in said memory means.

The selector can be operated manually by the user as a function of the visual verification performed by said user before proceeding with the treatment.

If one wishes to have a fully automated apparatus that requires no autonomous assessment on the part of the operator, the device 1 must comprise a sensor 8 intended to detect the light radiation reflected by the skin of the patient.

The control and management unit 3, as a function of the signal emitted by the sensor 8 following the detection of reflected light as a consequence of a test emission of light radiation by the at least one LED 4, is independently capable of setting the power of the light beam that will be emitted by the at least one LED 4 during the subsequent treatment by means of the apparatus for adjusting the electric power supply values of the at least one LED 4 as a function of the stored protocols.

Advantageously, the present invention solves the problems described earlier, proposing a device 1 for aesthetic treatments that allows to perform a uniform treatment of the entire skin surface that it faces.

This occurs by virtue of the specifically defined distance at which its LEDs 4 are located with respect to the skin of the patient.

Effectively, the device 1 is very safe for the patient, since it allows to perform treatments that take into account the specific characteristics of his skin, avoiding overexposures (excessive power of the light beam that strikes the skin), which might cause damage, or under exposures (insufficient power of the light beam that strikes the skin), which would produce no beneficial effect.

Positively, the device 1 for aesthetic treatments is simple to use for the operator since it requires (in the case of a non-automatic device 1) exclusively an estimate of the characteristics of the skin of the patient; in the case of a fully automatic device 1, no intervention on the part of the operator is required at all.

Validly, the device 1 can be manufactured at modest costs, since it is relatively simple to provide in practice and safe in application.

The invention thus conceived is susceptible of numerous modifications and variations, all of which are within the scope of the appended claims; all the details may further be replaced with other technically equivalent elements.

In the exemplary embodiments shown, individual characteristics, given in relation to specific examples, may actually be interchanged with other different characteristics that exist in other exemplary embodiments.

In practice, the materials used, as well as the dimensions, may be any according to the requirements and the state of the art.

What is claimed is:

1. A device for emitting light by means of LEDs of the continuous and pulsed type for aesthetic and therapeutic treatments, of the type comprising an electric power supply circuit which is connected to a respective external control and management unit, at least one LED controlled by said circuit and oriented toward a surface that is intended to face the skin of the patient in conditions for use, wherein the device has a platelike shape, said at least one LED being arranged on a laminar accommodation element that is arranged at a constant distance from the surface intended to face the skin of the patient in conditions for use, said electric circuit comprising at least one apparatus for adjusting the electric power supply values of said at least one LED controlled by said control and management unit in order to set the power of the light beam emitted by said at least one LED.

2. The device according to claim 1, wherein said electric circuit comprises an accommodation lamina for the respective components, including said at least one LED and said at least one adjustment apparatus, provided with conducting surface paths for the functional connection thereof.

3. The device according to claim 1, wherein said circuit is interposed between an external panel and a plate that lies on said surface intended to face the skin of the patient in conditions for use, said plate being transparent to the radiation emitted by said at least one LED.

4. The device according to claim 3, wherein at least one component chosen among said lamina, said external panel and said plate is made of flexible material.

5. The device according to claim 1, further comprising a sensor that is adapted to detect the light radiation reflected by the skin of the patient, said control and management unit, as a function of the signal emitted by said sensor, following the detection of reflected light as a consequence of a test emission of light radiation by the at least one LED, setting the power of the light beam emitted by said at least one LED through said apparatus for adjusting the electric power supply values of said at least one LED.

6. The device according to claim 1, wherein said at least one LED is a plurality of LEDs, distributed on said laminar accommodation element that is arranged at a constant distance from the surface that is intended to face the skin of the patient in conditions for use.

7. The device according to claim 1, wherein its shape and dimensions are complementary to those of a specific part of the human body for which it is intended.

8. An apparatus for aesthetic and therapeutic treatments of the type comprising a control and management unit, a treatment device, and at least one cable for connection to said treatment device provided with an electric power supply circuit for at least one LED that is oriented toward a surface of said device that is intended to face the skin of the patient in conditions for use, wherein said device is platelike and said at least one LED is arranged on a laminar accommodation element that is arranged at a constant distance from the surface that is intended to face the skin of the patient in conditions for use, said electric circuit comprising at least one apparatus for adjusting the electric power supply values of said at least one LED controlled by said control and management unit, in order to set the power level of the light beam emitted by said at least one LED as a function of operating parameters contained within memory means of said unit.

9. The apparatus for aesthetic and therapeutic treatments according to claim 8, wherein said control and management unit comprises a selector of a plurality of different treatment protocols related to specific skin problems and to specific skin types of the patient, said protocols being stored in said memory means.

10. The apparatus for aesthetic and therapeutic treatments according to claim 8, wherein said device comprises a sensor intended to detect the light radiation reflected by the skin of the patient, said control and management unit, as a function of the signal emitted by said sensor, following the detection of reflected light as a consequence of a test emission of light radiation by the at least one LED, setting the power level of the light beam emitted by said at least one LED by means of said apparatus for adjusting the electric power supply values of said at least one LED as a function of the stored protocols.
